(19) United States
(12) Patent Application Publication
(10) Pub. No.: US 2022/0047980 A1
(43) Pub. Date: Feb. 17, 2022

(54) SELF-CONTAINED SURGICAL SMOKE DETOXIFIER

(71) Applicant: Patientpocket LLC, CHERRY HILL, NJ (US)

(72) Inventor: Allen D. Seftel, Camden, NJ (US)

(73) Assignee: Patientpocket LLC, CHERRY HILL, NJ (US)

(21) Appl. No.: 17/399,838

(22) Filed: Aug. 11, 2021

Related U.S. Application Data
(60) Provisional application No. 63/064,593, filed on Aug. 12, 2020.

(51) Int. Cl. B01D 46/00; A61B 18/22

(52) U.S. Cl. CPC B01D 46/0049; A61B 2218/008; B01D 46/0043; A61B 18/22

(57) ABSTRACT

An integrated surgical smoke filtration and detoxification system. The system includes a housing that may include an interior chamber for detoxification and filtration performed by a detoxification system. The detoxification system may include a suction device and a filter and may be used to receive surgical smoke through a first portion of the chamber and exhaust detoxified surgical smoke through a second portion of the chamber.

### CROSS-REFERENCE TO RELATED APPLICATIONS

This application claims priority to U.S. Application Ser. No. 63/064,593, filed Aug. 12, 2020, which is hereby incorporated by reference in its entirety.

### BACKGROUND

Surgical smoke exposure, an unintended byproduct of laser, electrosurgical, and electrocautery-based procedures, has been a clinical concern for decades. During these procedures, energy-generating medical instruments can increase intracellular temperatures upwards to greater than 100° C., which can cause potentially dangerous and/or toxic particles or compounds to enter the air and be inhaled. This can include volatile organic compounds, respiratory irritants, biologically active particulates, potential carcinogens, and even harmful bacteria and viruses. For example, electrocautery-based procedures can involve using an electric cautery device to stop bleeding, but the cauterization can create surgical smoke as the cautery “burns” the tissue to stop the bleeding. The material given off in the generated smoke can be either harmful or infectious to staff and hospital personnel, which is undesirable.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIG. 1 shows a side, three-dimensional view of a device for storing a laser optical fiber, according to some embodiments of the present disclosure.

FIGS. 2A-2B show an integrated laser fiber safety holder and surgical smoke detoxification device, according to some embodiments of the present disclosure.

FIGS. 3A-3B show another integrated laser fiber safety holder and surgical smoke detoxification device, according to some embodiments of the present disclosure.

FIG. 4 shows a surgical smoke detoxification device, according to some embodiments of the present disclosure.

FIG. 5 shows another surgical smoke detoxification device, according to some embodiments of the present disclosure.

FIG. 6 shows another integrated laser fiber safety holder and surgical smoke detoxification device, according to some embodiments of the present disclosure.

FIG. 7 shows another integrated laser fiber safety holder and surgical smoke detoxification device, according to some embodiments of the present disclosure.

FIG. 8 shows various embodiments of a detoxification device as used in any of FIGS. 2A-7, according to some embodiments of the present disclosure.

The drawings are not necessarily to scale, or inclusive of all elements of a system, emphasis instead generally being placed upon illustrating the concepts, structures, and techniques sought to be protected herein.

### DETAILED DESCRIPTION

The following detailed description is merely exemplary in nature and is not intended to limit the invention or the applications of its use.

In recent times, the surgical smoke issue has been elevated to a new level due to COVID-19 and its potential for transmission via aerosolized viral particles contained in surgical smoke. This recent issue has been discussed in a recent article titled, “Resources for Smoke & Gas Evacuation During Open, Laparoscopic, and Endoscopic Procedures,” by SAGES Webmaster (Mar. 29, 2020), which can be found at https://www.sages.org/resources-smoke-gas-evacuation-during-open-laparoscopic-endoscopic-procedures/. Other research in this area includes: (1) Liu Y, Song Y, Hu X, Yan L, Zhu X. Awareness of surgical smoke hazards and enhancement of surgical smoke prevention among the gynecologists. J Cancer. 2019; 10(12):2788-2799. Published 2019 Jun. 2. doi:10.7150/jca.31464; (2) Georgesen C, Lipner, S R: Surgical smoke: Risk assessment and mitigation strategies J Am Acad Dermatol 2018; 79:746-55; (3) Mowbray N G, Ansell J, Horwood J, et al. Safe management of surgical smoke in the age of COVID-19 [published online ahead of print, 2020 May 3]. Br J Surg. 2020; 10.1002/bjs.11679. doi:10.1002/bjs.11679; and (4) Do M H, Minkis K, Petukhova T A, Lipner S R. Recommendations for personal protective equipment and smoke evacuation for dermatologic surgeries amid the COVID-19 crisis [published online ahead of print, 2020 May 15]. Dermatol Ther. 2020; e13592. doi:10.1111/dth.13592.

Current measures to deal with the surgical smoke issue can include the use of masks, the use of suction smoke evacuators, and operating room ventilation systems. However, standard surgical masks alone may not protect the wearer of the mask from aerosolized transmission due to leakage and a low filtration efficiency of small particles (approximately 90% effective). In addition, while N95 respirator masks can be marginally more efficient in filtering the smoke (approximately 95% effective for particles 0.3 microns and larger), they are still not fully capable of filtering all toxins from surgical smoke. Note, the RNA virus of COVID-19 is approximately 0.06 to 0.14 microns in size.

Smoke evacuation units are not always available in all healthcare settings, and those that are currently in use are expensive, stationary, loud, require off-side detoxification systems, and are generally cumbersome. Many evacuation units employ a hose and nozzle to be held by a surgical assistant to remove smoke from the operating field, but this can be tedious to coordinate as it involves multiple people. These systems also require large and additional equipment to provide the suction source to the hose and nozzle. Other evacuation units similarly employ a suction device connected to a hose and nozzle, but the hose and nozzle can be attached directly to a handheld laser device or surgical pen used by the surgeon. A shortcoming of these evacuators is that they can add unnecessary weight and affect the accuracy, focus, and attention of the surgeon while performing surgical procedures. A few evacuators utilize filters and suction devices that can rest stationary on the operating field, but they can involve similar shortcomings to previously mentioned evacuators: an off-site filtration, long, external tubing to bring smoke to the off-site location, processing, and filtration system are involved which can increase costs, complexity, and noise.

Currently, a best practice within the surgical field for mitigating possible infectious transmission during open laparoscopic and endoscopic procedures is to use a multi-faceted approach, which can include proper room filtration (e.g. HEPA, ULPA) and ventilation, appropriate personal protective equipment (PPE), and smoke evacuation devices with a suction and filtration.

Embodiments of the present disclosure relate to a portable and self-contained smoke evacuation and detoxification system for use on surgical operating fields. The evacuation and detoxification system can also be disposable and battery-operated. In some embodiments, the detoxification system can be incorporated into a single-use laser fiber safety holder, such as the device as described in U.S. application Ser. No. 13/577,781, now U.S. Pat. No. 8,917,965, which is herein incorporated by reference in its entirety. The integration can obviate current stationary, complex, multi-tiered smoke evacuation systems, which require separate electric powered suction and or filtration devices, electric outlets and off-site smoke detoxification and exhaust systems. In one embodiment, a small, portable, disposable device can include the integration of two safety systems, namely the prevention of accidental laser energy discharge and surgical smoke evacuation. The battery-operated nature can remove the need for nearby access to an electrical outlet, which can make the device more amenably to ICU environments or low-resource settings, such as army field hospitals and ambulatory surgery centers. In another embodiment, a small, portable, disposable device can simply include the surgical smoke evacuation assembly disclosed herein. Moreover, the surgical smoke evacuation assembly may comprise more than one configuration and technique for evacuating smoke.

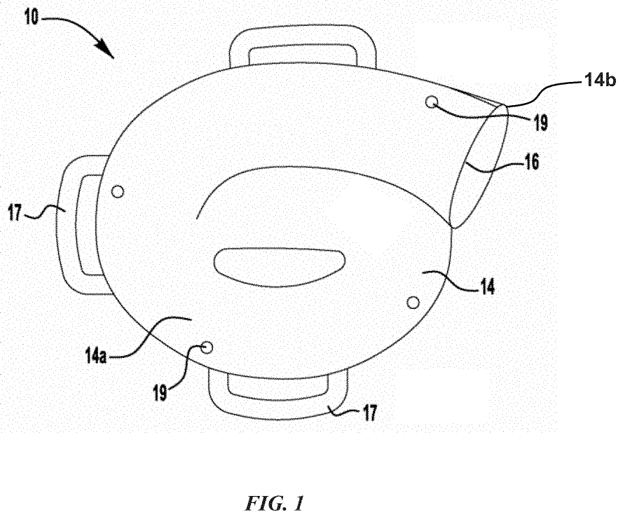

FIG. 1 shows a side, three-dimensional view of a device 10 for storing a laser optical fiber, according to some embodiments of the present disclosure. The device 10 can be the laser storage device disclosed by and patented in U.S. Pat. No. 8,917,965. The device 10 can temporarily store a laser optical fiber (not shown) as described in more detail in U.S. Pat. No. 8,917,965. Device 10 can include a storage housing 14 having an open end 16, which can receive a laser optical fiber. By temporarily disposing a laser optical fiber in storage housing 14, the free end (e.g., the end protruding from open end 16) can be protected from any potential source of contamination or potential breakage, and any accidentally discharged laser light from the laser optical fiber can be safely contained within the storage housing 14. In some embodiments, device 10 can be designed to be used during a medical procedure where a physician is using a laser optical fiber connected to a laser source (not shown) for emitting laser radiation. Device 10 may either be designed to be a one-use instrument, or to be sterilized and used a multitude of times. While the device 10 can be used during a medical procedure, it is within the terms of the invention to use it for safeguarding a laser optical fiber in any type of environment.

Device 10 can also include a number of ring-shaped protrusions 17 that can be attached to the top, side, and bottom. The ring-shaped protrusions 17 are used to attach device 10 to a patient, by means of a hook and loop (i.e., Velcro®) strap, flexible elastic, two-sided adhesive, or any other suit able attachment means. While device 10 is generally designed to be attached to the patient's leg, it is within the scope of the present invention to attach device 10 to any other part of the patient's body or to a non-patient site in proximity to the sterile or surgical field. In some embodiments, device 10 may rest on the operating field during a surgical procedure. In one or more embodiments, ring-shaped protrusions 17 are not used and the device includes a mechanism on a side of the housing 14, opposite the side shown in FIG. 1, to attach the device 10 to the surgical drape in proximity to the surgical site.

In some embodiments, device 10 can be constructed as a housing 14 with an open end 16. The storage housing 14 can be formed of two hollow storage housing sections 14a and 14b, which are the mirror image of each other. When housing sections 14a and 14b are joined together, they can form a spiral-shaped channel within the interior. While screws 19 are indicated as the means to join the housing sections 14a and 14b together, it is within the terms of the invention to join them in any desirable way, such as with an adhesive. The storage housing 14 can be constructed of a variety of materials, such as but not limited to, for example, plastic, silicone, polymers and aluminum.

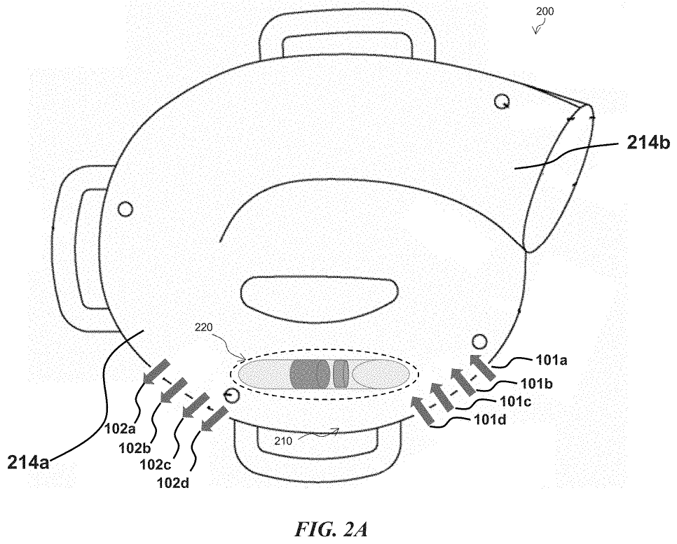

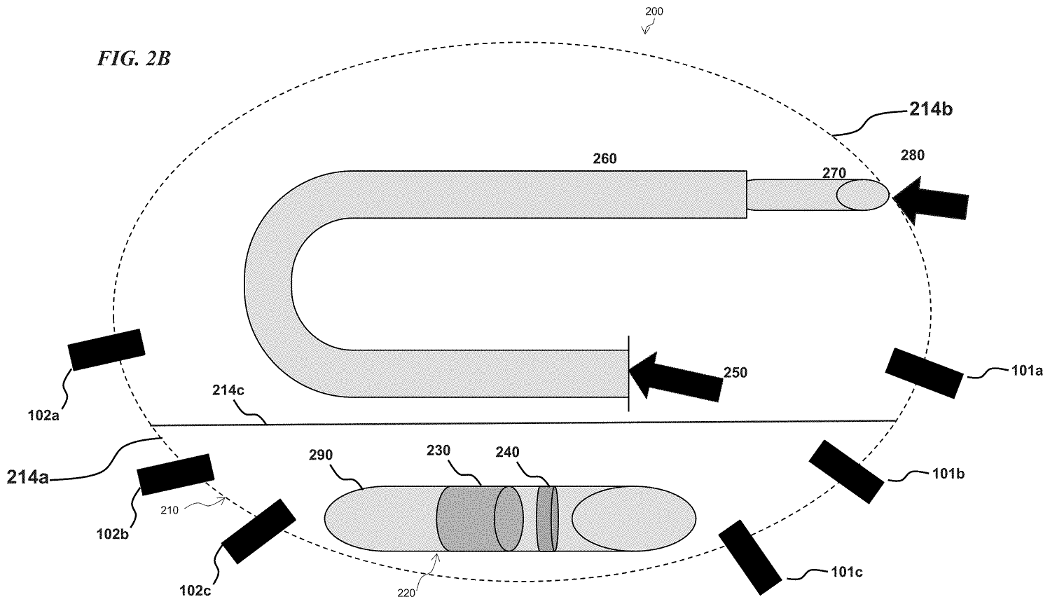

FIGS. 2A-2B show an integrated laser fiber safety holder and surgical smoke detoxification device 200, according to some embodiments of the present disclosure. FIG. 2A shows the outside of the device 200, but also shows a part of an inner portion of the device 200 within the dashed lines. Details of the inner portion are shown in FIG. 2B. The device 200 of FIGS. 2A and 2B can be the device 10 of FIG. 1 adapted to include an integrated surgical smoke filtration and detoxification system 220 according to the disclosed principles. The lower half of housing 14 (housing section 214a) can include an interior chamber 210 for detoxification and filtration performed by detoxification system 220. The device 200 also includes an upper half 214b. In some embodiments, the interior chamber 210 can be separate and or sealed off from the interior spiral-shaped chamber (as shown by line 214c) as described with respect to FIG. 1 and partly illustrated in FIG. 2B. The interior spiral-shaped chamber can include a large tubing 260 connected to a small tubing 270, which are adjacent to an insertion post 280 (e.g., twist latch clamp). In some embodiments, the interior spiral-shaped chamber can be sealed at the end 250.

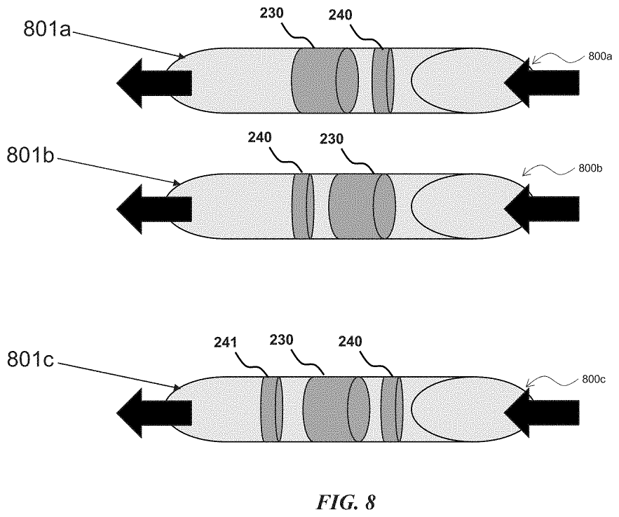

The detoxification system 220 may reside between a plurality of entry holes 101a-101d and a plurality of exit holes 102a-102d, although only entry holes 101a-102c and exit holes 101a-101c are shown in FIG. 2B. In some embodiments, as shown in FIG. 2B, the detoxification system 220 may include tubing 290, along with an internal filter 240, such as e.g., a HEPA filter. It should be appreciated that the internal filter 240 is not limited to being a HEPA filter and that any suitable filter may be used as internal filter 240 (or any other filter discussed herein) including e.g., an ultra low particulate air (ULPA) filter, allergen, bacteria and or virus filters. In some embodiments, the detoxification system 220 can also include a device 230 for increasing suction, such as a battery-operated suction pump or fan, herein referred to as suction device 230. The filter 240 and the suction device 230 can reside within the tubing 290. In addition, although FIGS. 2A and 2B show an embodiment where the filter 240 is positioned to receive the smoke prior to suction device 230, the disclosed principles are not limited to such an order. Additional orientations are discussed in relation to FIG. 8. The placement of the filter 240 can provide detoxification of surgical smoke very close to the source (e.g., a body being operated on). In the illustrated embodiment, surgical smoke can enter the interior chamber on the right side of the device (according to the orientation shown in FIGS. 2A and 2B) via the plurality of entry holes 101a-101c, be suctioned through the detoxification system 220, and exit the interior chamber on the left side of the device through the plurality of exit holes 102a-102c. This can provide detoxified and filtered smoke to be exhausted on-site. In some embodiments, any number of holes can be used (e.g., 2, 3, 4, 5 or more). In some embodiments, any radius of hole can be used, such as 1 cm, 2 cm, or smaller (e.g., on the scale of millimeters). Although the illustrated example shows holes 101a, 102a formed in the upper half of the housing 14, one or more embodiments disclosed herein may confine the holes to the region covering the interior chamber 210 only.

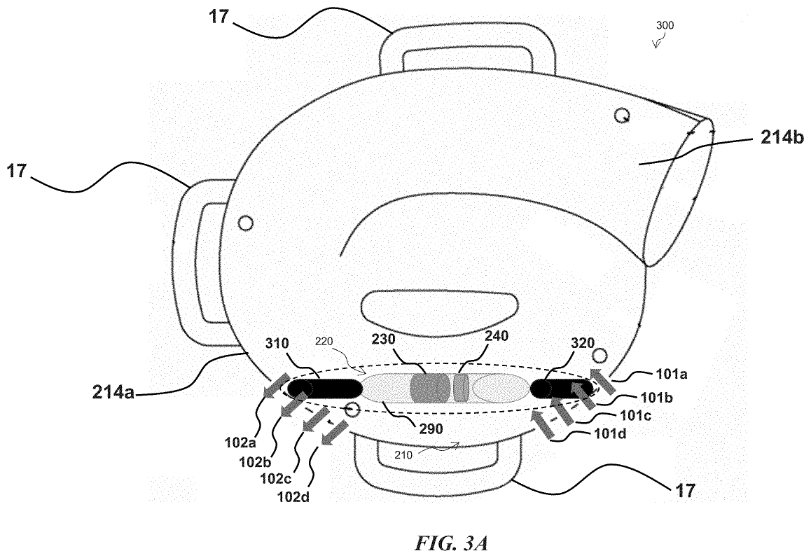

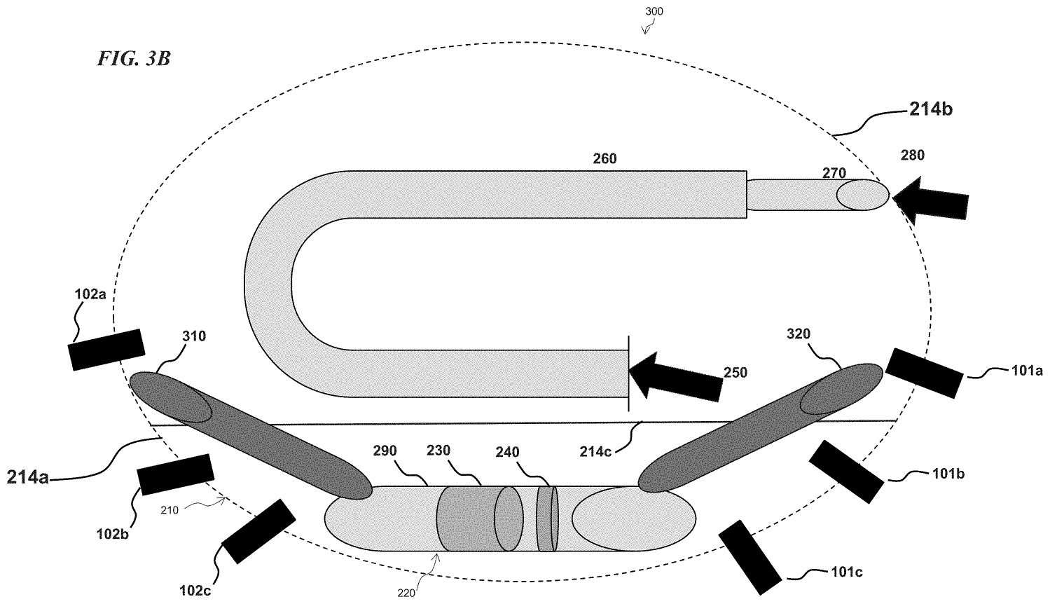

FIGS. 3A-3B show another integrated laser fiber safety holder and surgical smoke detoxification device 300, according to some embodiments of the present disclosure. FIG. 3A shows the outside of the device 200, but also shows a part of an inner portion of the device 200 within the dashed lines. Details of the inner portion are shown in FIG. 3B. The device 300 of FIGS. 3A and 3B can be the same or similar to the device 200 of FIGS. 2A and 2B, with the exception of added internal tubes 310 and 320 within interior chamber 210 connecting the plurality of entryways from the entry holes 101a-101c and exit-ways to the exit holes 102a-102d to the tubing 290 of the detoxification system 220. In some embodiments, this can facilitate more efficient suction of air into the detoxification system 220 than simply placing the system 220 in the chamber. In some embodiments, any of the devices 200, 300, 400, 500, 600, 700 in FIGS. 2A-7 can rest or be adhered to a surgical field to provide on-site detoxification and exhaust of surgical smoke. In some embodiments, any of the devices 200, 300, 400, 500, 600, 700 in FIGS. 2A-7 can be disposable. Although the illustrated example shows holes 101a, 102a and a portion of the internal tubes 310, 320 being formed in the upper half of the housing 14, one or more embodiments disclosed herein may confine the holes and the internal tubing to the region covering the interior chamber 210 only.

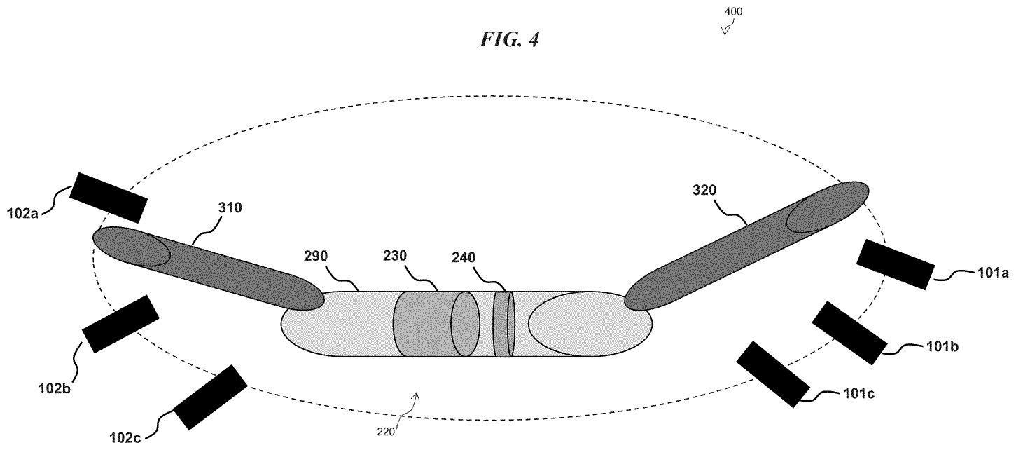

FIG. 4 shows a surgical smoke detoxification device 400, according to some embodiments of the present disclosure. The device 400 can include the suction and detoxification assembly 220, as described in relation to FIGS. 2A, 2B, 3A and 3B, as a standalone device. In other words, device 400 can exist as an independent device without being integrated into a laser fiber safety holder device (shown in FIG. 1). Device 400 can be used in a similar manner as the previously described devices (e.g., devices 200 and 300). For example, device 400 can include a mechanism for attaching to an operating field (not shown), such as Velcro®, adhesives, etc., tubing 290, an internally located battery-powered suction device 230, and internal filter 240 contained within detoxification system 220. The system 200 can receive smoke via a plurality of entry holes 101a-101c on one side of the device's 400 housing and can provide filtered exhaust via the plurality of exit holes 102a-102c on the opposite side of the device's 400 housing. In the illustrated embodiment, surgical smoke can be suctioned in through the entry holes 101a-101c on the right side of device 400 (according to the orientation in FIG. 4) via suction device 230, detoxified via the internal filter 240, and exhausted through the plurality of exit holes 102a-102c. As shown in FIG. 4, device 400 can include internal tubing 310 and 320 connecting one or more of the plurality of entry holes 101a-101c and one or more of the plurality of exit holes 102a-102c with the tubing 290 forcing smoke through the internal filter 240 and suction device 230, which can facilitate more efficient suction and filtering, similar to as described in relation to the device of FIGS. 3A-3B.

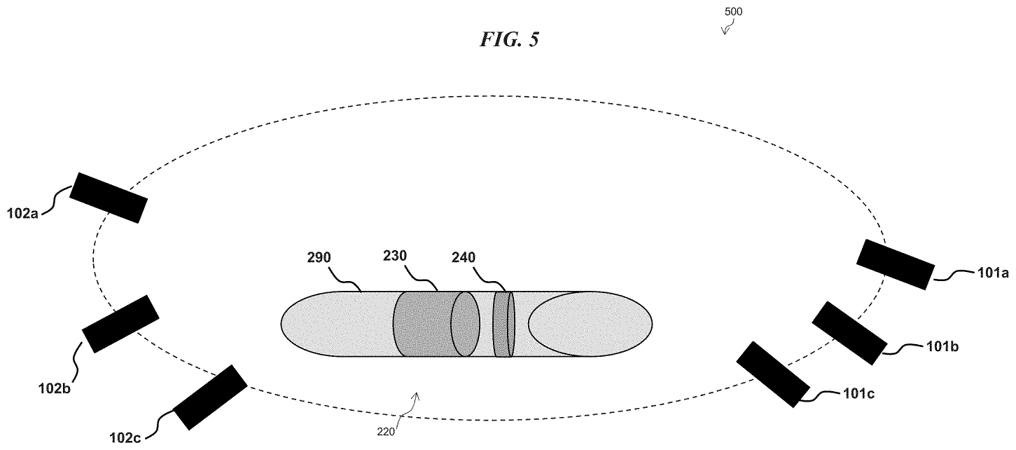

FIG. 5 shows another surgical smoke detoxification device 500, according to some embodiments of the present disclosure. The device 500 can be the same as or similar to the device 400 of FIG. 4, with the exception that there is no internal tubing (e.g., tubing 310 and 320 of FIG. 4) connecting the internal filter 240 and suction device 230 within detoxification system 220 with the entry holes 101a-101c and exhaust/exit holes 102a-102c. In the illustrated embodiment, surgical smoke can be suctioned in through the plurality of holes 101a-101c on the right side of the device 500, passed through the internal filter 240 and tubing 290 via the battery-power suction device 230, and exhausted through the left plurality of holes 102a-102c. Device 500 can also be disposable and can be adhered to an operating site in order to provide on-site detoxification and exhaust of surgical smoke.

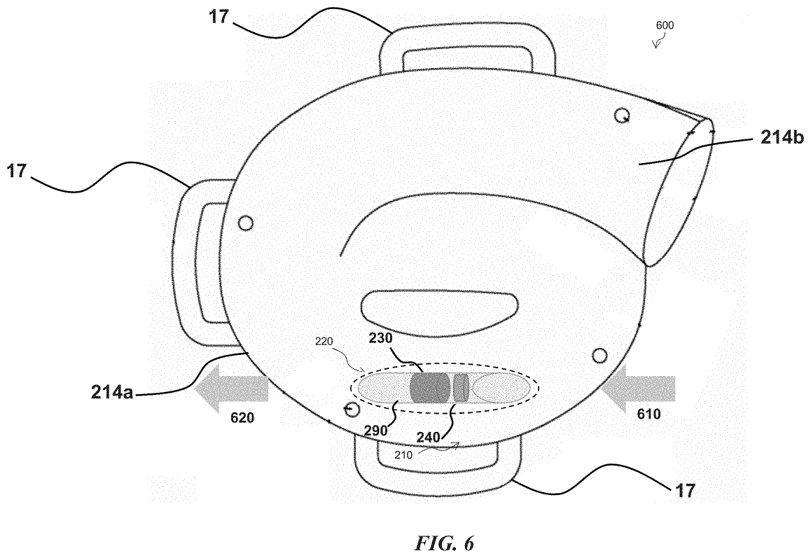

FIG. 6 shows another integrated laser fiber safety holder and surgical smoke detoxification device 600, according to some embodiments of the present disclosure. Device 600 can be the same as or similar to the integrated laser fiber safety holder and surgical smoke detoxification device 200 of FIGS. 2A-2B, with the exception that device 600 only contains a single hole on either side of the housing (e.g., entry hole 610 and exit hole 620) that allows the suction device and filter to detoxify surgical smoke obtained from a surgical site. Device 600 can include a battery-operated suction device 230 and an internal filter 240 within tubing 290, forming detoxification system 220 as discussed above. In some embodiments, each hole 610 and 620 may be round and have a size larger than a hole that would be included in device 200. It should be appreciated that each hole 610 and 620, both in relation to FIG. 6 and the rest of the disclosure, is not restricted to being round and may comprise other cross-sectional shapes, such as rectangular, oval, triangular, hexagonal, octagonal, to name a few. In some embodiments, the holes 610 and 620 on each side of the device 600 may be the same shape and size, although in other embodiments they may be different. As described with respect to FIGS. 2A-5, device 600 may rest on or be attached to the operating site by various means and may detoxify surgical smoke and exhaust the detoxified smoke near the source without the need for long, external tubing or large evacuators.

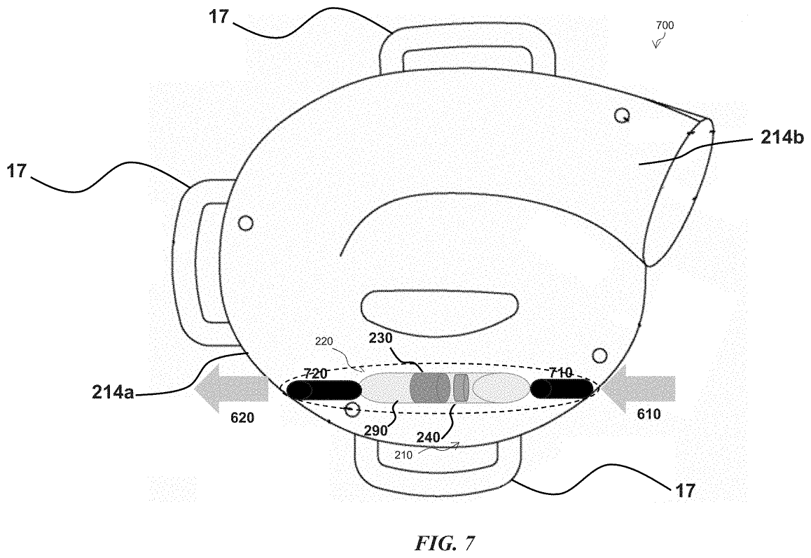

FIG. 7 shows another integrated laser fiber safety holder and surgical smoke detoxification device 700, according to some embodiments of the present disclosure. Device 700 may be the same as or similar to device 600 of FIG. 6, with the exception of the added internal tubing 710 and 720 to facilitate suction and detoxification, such as described above with respect to FIGS. 3A, 3B and 4. Device 700 can include a battery-operated suction device 230 and an internal filter 240 within tubing 290, forming a detoxification system 220 located within internal chamber 210. The internal tubing 710 and 720 may be attached to tubing 290 and can also be positioned within the interior chamber 210.

FIG. 8 shows various embodiments of a detoxification device as used in any of FIGS. 2A-7, according to some embodiments of the present disclosure. Specifically, FIG. 8 shows various embodiments of detoxification system 220 as described in FIGS. 2A-7 and various arrangements of filters and suction devices. An example suction device can be a fan or other battery-powered device, such as the air flow generator as described in U.S. Pat. No. 5,709,675. One example of detoxification system 220 can be system 800a that includes tubing 801a, where surgical smoke passes through an internal filter 240 first before proceeding through the suction device 230 and being exhausted on the left side. Another example of detoxification system 220 can be system 800b that includes tubing 801b, where the surgical smoke passes through the suction device 230 prior to being filtered by the internal filter 240. System 800c can be another example of detoxification system 220 that includes tubing 801c and can include filters 240 and 241 on both sides of the suction device 230. As with other embodiments, the filters 240, 241 may be HEPA or other filters discussed above.

In any embodiment, the chamber, casing, tube connections will be sealed to prevent smoke from seeping out of the device. Any sealant material suitable for blocking smoke may be used. One or more embodiments disclosed herein may be useful for all surgical procedures, and would be particularly useful for ENT surgical cases, anesthesia surgical procedural cases, thoracic surgical cases, dermatologic surgical procedural cases, ob-gyn cases, and similar cases wherein the provider may have a tendency to have a higher risk of exposure to aerosolized particles. Other settings in which one or more of the disclosed embodiments may be used include physicians' offices, ambulatory surgery centers, and field operation units wherein a portable, self-contained surgical smoke evacuation unit would be advantageous over current systems.

In one or more embodiments, a small extension tubing may be added to and extend from the device 200, 300, 400, 500, 600, 700 so that the device can suction smoke off the operating room field without having another device too close to the field and becoming intrusive to the equipment and operating personnel. In one or more embodiments, a flap or other attachment mechanism may be added to the housing of the device 200, 300, 400, 500, 600, 700 to attach the device to a surgical stand.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter. It is important, therefore, that the claims be regarded as including such equivalent constructions insofar as they do not depart from the spirit and scope of the disclosed subject matter.

Although the disclosed subject matter has been described and illustrated in the foregoing illustrative embodiments, it is understood that the present disclosure has been made only by way of example, and that numerous changes in the details of implementation of the disclosed subject matter may be made without departing from the spirit and scope of the disclosed subject matter.

1.-10. (canceled)

11. A disposable surgical smoke detoxification device comprising:
a housing;
an interior chamber within the housing, the interior chamber comprising a first end and a second end;
a suction device and a filter within the interior chamber, the suction device being configured to suction surgical smoke through the filter;
at least one first hole located on the first end of the interior chamber for receiving surgical smoke from a surgical site; and
at least one second hole located on the second end of the interior chamber for exhausting detoxified surgical smoke.

12. The disposable surgical smoke detoxification device of claim 11, wherein the housing comprises a tube portion configured to receive the surgical smoke from the surgical site.

13. The disposable surgical smoke detoxification device of claim 11, wherein the filter is a HEPA filter.

14. The disposable surgical smoke detoxification device of claim 11, wherein the interior chamber comprises tubing that contains the suction device and the filter.

15. The disposable surgical smoke detoxification device of claim 14, wherein the interior chamber comprises:
a first internal tube connecting the at least one first hole to a first end of the tubing; and
a second internal tube connecting the at least one second hole to a second end of the tubing.

16. The disposable surgical smoke detoxification device of claim 11, wherein the suction device comprises at least one of a fan or a pump.

17. The disposable surgical smoke detoxification device of claim 11, wherein the at least one first hole and the at least one second hole are circular, rectangular, triangular, ovular, hexagonal, or octagonal in shape.

18. The disposable surgical smoke detoxification device of claim 11, wherein the surgical smoke is received by the filter prior to being received by the suction device.

19. The disposable surgical smoke detoxification device of claim 11, wherein the surgical smoke is received by the suction device prior to being received by the filter.

20. The disposable surgical smoke detoxification device of claim 11 comprising a filter on both sides of the suction device.

21. An integrated smoke detoxification and laser optical fiber storage device comprising:
a housing;
an interior chamber within the housing, the interior chamber comprising a first end and a second end;
a suction device and a filter within the interior chamber, the suction device being configured to suction surgical smoke through the filter;
a first hole located on the first end of the interior chamber for receiving surgical smoke from a surgical site; and
a second hole located on the second end of the interior chamber for exhausting detoxified surgical smoke.

22. The integrated smoke detoxification and laser optical fiber storage device of claim 21, wherein the housing comprises a tube portion configured to receive the surgical smoke from the surgical site.

23. The integrated smoke detoxification and laser optical fiber storage device of claim 21, wherein the filter is a HEPA filter.

24. The integrated smoke detoxification and laser optical fiber storage device of claim 21, wherein the interior chamber comprises tubing that contains the suction device and the filter.

25. The integrated smoke detoxification and laser optical fiber storage device of claim 24, wherein the interior chamber comprises:
a first internal tube connecting the at least one first hole to a first end of the tubing; and
a second internal tube connecting the at least one second hole to a second end of the tubing.

26. The integrated smoke detoxification and laser optical fiber storage device of claim 21, wherein the suction device comprises at least one of a fan or a pump.

27. The integrated smoke detoxification and laser optical fiber storage device of claim 21, wherein the first hole and the second hole are circular.

28. The integrated smoke detoxification and laser optical fiber storage device of claim 21, wherein the surgical smoke is received by the filter prior to being received by the suction device.

29. The integrated smoke detoxification and laser optical fiber storage device of claim 21, wherein the surgical smoke is received by the suction device prior to being received by the filter.

30. The integrated smoke detoxification and laser optical fiber storage device of claim 21 comprising a filter on both sides of the suction device.
